(19) United States
(12) Patent Application Publication
(10) Pub. No.: US 2024/0131366 A1
(43) Pub. Date: Apr. 25, 2024

(54) DEVICE AND METHOD FOR INDUCING A WIDE AREA OF STABLE CAVITATION AND CONTROLLING FOR INERTIAL CAVITATION

(71) Applicant: Adenocyte Ltd., Jerusalem (IL)

(72) Inventors: Dan ADAM, Haifa (IL); Kyle MORRISON, Bothell, WA (US)

(73) Assignee: Adenocyte Ltd., Jerusalem (IL)

(21) Appl. No.: 18/404,957

(22) Filed: Jan. 5, 2024

Related U.S. Application Data
(63) Continuation of application No. PCT/IL2022/050726, filed on Jul. 6, 2022.
(60) Provisional application No. 63/218,558, filed on Jul. 6, 2021.

(51) Int. Cl. A61N 7/00

(52) U.S. Cl. CPC A61N 7/00; A61N 2007/0039; A61N 2007/0052

(57) ABSTRACT

A method and apparatus for producing an organ-sized area of stable microbubble cavitation including insonating an organ of a patient using Low Intensity Non-Focused Ultrasound (LINFU) at a first setting, monitoring the organ to detect presence of desired stable cavitation microbubble resonance and presence of unwanted inertial cavitation, when the presence of stable cavitation microbubble resonance is not detected then adjusting insonation parameters so as to increase the level of insonation, and when inertial cavitation is detected then adjusting the insonation parameters so as to decrease the level of insonation. Related apparatus and methods are also described.

### RELATED APPLICATIONS

This application is a Continuation of PCT Patent Application No. PCT/IL2022/050726 having International filing date of Jul. 6, 2022, which claims the benefit of priority under 35 USC § 119(e) of U.S. Provisional Patent Application No. 63/218,558 filed on Jul. 6, 2021. The contents of the above applications are all incorporated by reference as if fully set forth herein in their entirety.

### FIELD AND BACKGROUND OF THE INVENTION

The present disclosure, in some embodiments thereof, relates to a Low Intensity Non-Focused Ultrasound (LINFU) device for creating a wide area of stable cavitation through resonance of intravenously introduced microbubbles inside a pancreas or another organ sized area of the body, and, more particularly, but not exclusively, to such a device which includes controlling production of unwanted inertial cavitation. In some embodiments, the device may serve to induce exfoliation inside the body. In some embodiments, the device may serve to induce sonoporation.

Additional background art includes:
International Patent Application Publication Number WO 2019/183623 of Rutenberg et al.;
International Patent Application Publication Number WO 2021/042042 of Adam et al.;
An article titled “Monitoring and control of inertial cavitation activity for enhancing ultrasound transfection: The SonInCaRe project” by Inserraa, P. Labellea, C. Der Loughian, J.-L. Leec, M. Fouqueraya, J. Ngoa, A. Poizata, C. Desjouya, B. Munteanud, C.-W. Loc, C. Vanbellea, J.-P. Rieub, W.-S. Chenc, J.-C. Bera, published in ScienceDirect www(dot)sciencedirect(dot)com IRBM 35 (2014) 94-99;
An article title “A human clinical trial using ultrasound and microbubbles to enhance gemcitabine treatment of inoperable pancreatic cancer” by Georg Dimcev ski, Spiros Kotopoulis, Tormod Blanes, Dag Hoem, Jan Schjott, Bjorn Tore Gjertsen, Martin Biermann, Anders Molven, Halfdan Sorbye, Emmet McCormack, Michiel Postema, Odd Helge Gilja, published in Journal of Controlled Release 243 (2016) 172-181; and
An article titled “SONOPORATION OF ADHERENT CELLS UNDER REGULATED ULTRASOUND CAVITATION CONDITIONS” by Pauline Muleki Seya, Manuela Fouqueray, Jacqueline Ngo, Adrien Poizat, Claude Inserra, and Jean-Christophe Bera, published in Ultrasound in Med. & Biol., Vol. 41, No. 4, pp. 1008-1019, 2015

The disclosures of all references mentioned above and throughout the present specification, as well as the disclosures of all references mentioned in those references, are hereby incorporated herein by reference.

### SUMMARY OF THE INVENTION

The present disclosure, in some embodiments thereof, relates to a Low-Intensity Non-Focused Ultrasound (LINFU) method and device for creating a wide area of stable cavitation through resonance of intravenously introduced microbubbles inside the entire pancreas or another organ sized area of the body, and, more particularly, but not exclusively, to such a device which includes controlling production of unwanted inertial cavitation. In some embodiments, the device may serve to induce exfoliation inside the body. In some embodiments, the device may serve to induce sonoporation.

According to an aspect of some embodiments of the present disclosure there is provided a method for producing an organ-sized area of stable microbubble cavitation including insonating an organ of a patient using Low Intensity Non-Focused Ultrasound (LINFU) at a first setting, monitoring the organ to detect presence of desired stable cavitation microbubble resonance and presence of unwanted inertial cavitation, when the presence of stable cavitation microbubble resonance is not detected then adjusting insonation parameters so as to increase the level of insonation, and when inertial cavitation is detected then adjusting the insonation parameters so as to decrease the level of insonation.

According to some embodiments of the disclosure, monitoring the pancreas includes producing ultrasound images of the pancreas, and monitoring for microbubble resonance by detecting the microbubble resonance in the ultrasound images.

According to some embodiments of the disclosure, detecting the microbubble resonance in the ultrasound images includes performing image analysis of the ultrasound images.

According to some embodiments of the disclosure, monitoring the pancreas includes monitoring for inertial cavitation by cavitation detectors.

According to an aspect of some embodiments of the present disclosure there is provided a method for producing an organ-sized area of stable microbubble cavitation including insonating a pancreas of a patient using Low-Intensity Non-Focused Ultrasound (LINFU) at a first setting, monitoring the patient for inertial cavitation, identifying a depth of the inertial cavitation, automatically adjusting insonation when the depth of the inertial cavitation is greater than an anterior surface of the pancreas.

According to some embodiments of the disclosure, the first setting includes a setting for exfoliation, using a Mechanical Index (MI) in a range of 0.3 to 0.8.

According to some embodiments of the disclosure, the first setting includes a setting for sonoporation, using a Mechanical Index (MI) in a range of 1.3-1.9.

According to some embodiments of the disclosure, the adjusting insonation includes controlling insonation to avoid causing tissue damage.

According to some embodiments of the disclosure, the monitoring includes using a plurality of cavitation detectors to determine the depth of the inertial cavitation, and the identifying a depth of the inertial cavitation includes correlating two or more ultrasound signals received from the cavitation detectors.

According to some embodiments of the disclosure, the adjusting insonation includes adjusting insonation to reduce detected cavitation.

According to some embodiments of the disclosure, further including measuring temperature of an ultrasound probe, and adjusting insonation when temperature at a subject skin rises above a threshold temperature.

According to some embodiments of the disclosure, further including estimating temperature at the pancreas, and adjusting insonation when estimated temperature at the pancreas rises above a threshold temperature.

According to some embodiments of the disclosure, the adjusting insonation includes adjusting duration of ultrasound pulses.

According to some embodiments of the disclosure, the adjusting insonation includes adjusting duty cycle of ultrasound pulses.

According to some embodiments of the disclosure, the insonating the pancreas of a patient includes insonating a first portion of the pancreas, and the automatically adjusting insonation includes steering the insonation to a second, different, portion of the pancreas.

According to some embodiments of the disclosure, the adjusting insonation includes adjusting amplitude of the insonation.

According to some embodiments of the disclosure, the adjusting insonation includes adjusting amplitude of a group of ultrasound transducers by a similar factor.

According to some embodiments of the disclosure, the adjusting insonation includes adjusting amplitude of at least one ultrasound transducer by a different factor than at least one other ultrasound transducer.

According to some embodiments of the disclosure, the adjusting insonation includes adjusting frequency of at least one ultrasound transducer.

According to some embodiments of the disclosure, the adjusting insonation includes adjusting frequency of at least one ultrasound transducer by a different factor than at least one other ultrasound transducer.

According to some embodiments of the disclosure, the adjusting insonation includes adjusting relative phase of at least one ultrasound transducer by a different amount than at least one other ultrasound transducer.

According to some embodiments of the disclosure, the adjusting insonation includes adjusting a direction of an ultrasound beam formed by a plurality of ultrasound transducers.

According to some embodiments of the disclosure, the adjusting insonation includes adjusting a focus of an ultrasound beam formed by a plurality of ultrasound transducers.

According to some embodiments of the disclosure, further including cooling ultrasound transducers which perform the insonating.

According to an aspect of some embodiments of the present disclosure there is provided a Low Intensity Non-Focused Ultrasound (LINFU) device including an ultrasound probe including an ultrasound transducer, a cavitation detector, an electronics unit for adjusting insonation of the ultrasound probe, and a processor for analyzing signals from the cavitation detector and controlling the insonation using the electronics unit.

According to some embodiments of the disclosure, the probe further includes an ultrasound imaging probe.

According to some embodiments of the disclosure, the probe is shaped to fit between a patient's ribs, below the patient's sternum.

According to some embodiments of the disclosure, the device includes a plurality of cavitation detectors.

According to some embodiments of the disclosure, the device includes a plurality of ultrasound transducers.

According to some embodiments of the disclosure, the plurality of ultrasound transducers are arranged in a random pattern.

According to some embodiments of the disclosure, the processor is configured to determine a depth of cavitation detected by the cavitation detector.

According to some embodiments of the disclosure, the processor is configured to determine a three dimensional location of cavitation detected by the cavitation detector.

According to some embodiments of the disclosure, further including a component for removing heat from the ultrasound probe.

According to some embodiments of the disclosure, further including a belt for attaching to a subject's body.

According to some embodiments of the disclosure, further including a temperature sensor for measuring temperature at a subject's body.

According to some embodiments of the disclosure, further including a temperature sensor for measuring temperature at the ultrasound probe.

According to an aspect of some embodiments of the present disclosure there is provided a system for producing an organ-sized area of stable microbubble cavitation including a device as described herein, and a user interface configured for entering parameters related to producing stable microbubble resonance while avoiding inertial cavitation.

According to some embodiments of the disclosure, the user interface is configured for entering physical parameters related to a subject planned for exfoliation.

According to some embodiments of the disclosure, the user interface is configured for entering physical parameters related to a subject planned for sonoporation.

According to some embodiments of the disclosure, the system includes communication with medical database for obtaining subject data.

According to an aspect of some embodiments of the present disclosure there is provided a method for producing an organ-sized area of stable microbubble cavitation including insonating a pancreas of a patient using Low Intensity Non-Focused Ultrasound (LINFU) at a first setting, determining temperature produced by the insonating, automatically adjusting insonation when the temperature exceeds a threshold temperature.

According to some embodiments of the disclosure, the first setting includes a setting for exfoliation.

According to some embodiments of the disclosure, the first setting includes a setting for sonoporation.

According to some embodiments of the disclosure, the determining temperature includes measuring temperature of an ultrasound probe used for the insonating.

According to some embodiments of the disclosure, the determining temperature includes measuring temperature of a subject's skin at a location of the insonating.

According to some embodiments of the disclosure, the determining temperature includes estimating temperature of the pancreas.

According to some embodiments of the disclosure, the adjusting insonation includes adjusting amplitude of the insonation.

According to some embodiments of the disclosure, the adjusting insonation includes adjusting duration of ultrasound pulses.

According to some embodiments of the disclosure, the adjusting insonation includes adjusting duty cycle of ultrasound pulses.

According to some embodiments of the disclosure, the insonating the pancreas of a patient includes insonating a first portion of the pancreas, and the automatically adjusting insonation includes steering the insonation to a second, different, portion of the pancreas.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present disclosure may be embodied as a system, method or computer program product. Accordingly, some embodiments of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present disclosure may take the form of a computer program product embodied in one or more computer readable medium(s) having computer readable program code embodied thereon. Implementation of the method and/or system of some embodiments of the disclosure can involve performing and/or completing selected tasks manually, automatically, or a combination thereof. Moreover, according to actual instrumentation and equipment of some embodiments of the method and/or system of the disclosure, several selected tasks could be implemented by hardware, by software or by firmware and/or by a combination thereof, e.g., using an operating system.

For example, hardware for performing selected tasks according to some embodiments of the disclosure could be implemented as a chip or a circuit. As software, selected tasks according to some embodiments of the disclosure could be implemented as a plurality of software instructions being executed by a computer using any suitable operating system. In an exemplary embodiment of the disclosure, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as controlling production of inertial cavitation, or detecting microbubbles in an ultrasound image, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIG. 1 is a simplified illustration of a device constructed according to an example embodiment, strapped to a torso;

FIG. 2A is a simplified block diagram illustration of a Low Intensity Non-Focused Ultrasound (LINFU) device for inducing a wide area of stable microbubble cavitation according to an example embodiment;

FIG. 2B is a simplified illustration of a system 200 for inducing a wide area of stable microbubble cavitation inside a body according to an example embodiment;

FIG. 3A is a simplified block diagram illustration of a system for Low Intensity Non-Focused Ultrasound (LINFU) for inducing a wide area of stable microbubble cavitation inside a body according to an example embodiment;

FIG. 3B is a simplified illustration of insonating a first portion of a target organ, followed by insonating a second portion of the target organ, according to an example embodiment;

FIGS. 4A and 4B are simplified illustrations of an ultrasound probe according to an example embodiment;

FIGS. 4C and 4D are simplified illustrations of an ultrasound probe according to an example embodiment;

FIGS. 4E and 4F are simplified illustrations of an ultrasound probe according to an example embodiment;

FIG. 5A is a three-dimensional graph of pressure produced by insonation as a function of two-dimensional location at a depth Z in a body, according to an example embodiment;

FIG. 5B is a graph of pressure produced by insonation as a function of one-dimensional location, according to an example embodiment;

FIG. 5C is a three-dimensional graph of pressure produced by insonation as a function of depth Z in a body, according to an example embodiment;

FIG. 6 is a simplified illustration of an ultrasound probe with a random scattering of ultrasound transducers according to an example embodiment;

FIGS. 7A, 7B and 7C are simplified illustrations of ultrasound probes with an arrangement of ultrasound transducers and cavitation detectors according to example embodiments;

FIG. 8 is a simplified illustration of a volume of insonation according to an example embodiment;

FIG. 9A is a simplified illustration of a heat conducting cover for an ultrasound probe according to an example embodiment;

FIG. 9B is a simplified illustration of a heat conducting cover and an attachment belt for an ultrasound probe according to an example embodiment;

FIG. 9C is a simplified illustration of an attachment belt and an ultrasound probe or probe cover according to an example embodiment;

FIG. 10 is a simplified flow chart illustration of a method for exfoliating a pancreas according to an example embodiment; and

FIG. 11 is a simplified illustration of controlling a level of insonation according to an example embodiment.

### DESCRIPTION OF SPECIFIC EMBODIMENTS OF THE INVENTION

The present disclosure, in some embodiments thereof, relates to a Low Intensity Non-Focused Ultrasound (LINFU) device for creating a wide area of stable microbubble cavitation inside a body, and, more particularly, but not exclusively, to such a device which includes controlling production of unwanted inertial cavitation. In some embodiments, the wide area of stable microbubble cavitation may serve for exfoliation inside the body. In some embodiments, the wide area of stable microbubble cavitation may serve for sonoporation.

The term LINFU is used in the present specification and claims for Low Intensity Non-Focused Ultrasound, and should be understood to imply that the intensity is less than what would be required to generate inertial cavitation and that ultrasound intensity is not otherwise limited.

Stable cavitation typically implies linear oscillation.

Sonoporation may use higher ultrasound intensities than used in exfoliation—thus possibly producing non-linear oscillations. Methods, devices, and systems described herein are designed to reduce and/or avoid implosion, or inertial cavitation.

Some embodiments described herein control levels of insonation, in some embodiments on a wide area simultaneously (e.g. a whole organ/pancreas).

In some embodiments, controlling the levels of insonation operate at low intensities for exfoliation.

In some embodiments, controlling the levels of insonation operate at levels that may cause non-linear oscillations for sonoporation.

In some embodiments, in both exfoliation and sonoporation, controlling the levels of insonation operate to avoid damage and/or reduced efficacy caused by implosion (inertial cavitation).

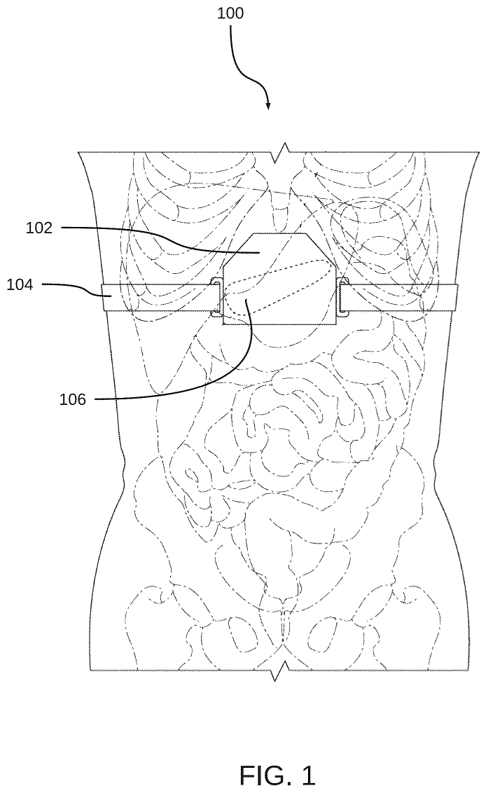

For purposes of better understanding some embodiments of the present disclosure, reference is first made to the construction and operation of a device as illustrated in FIG. 1.

FIG. 1 is a simplified illustration of a device constructed according to an example embodiment, strapped to a torso.

FIG. 1 shows a torso 100, within which are shown outlines of inner organs, and a device 102 strapped 104 to the torso 100.

A non-limiting example of an inner organ is a pancreas 106. The device 102 is shown in FIG. 1 placed on the torso above the pancreas 106.

The device 102 is a LINFU probe as described, for example, in above-mentioned International Patent Application Publication Number WO 2021/042042 of Adam et al.

By way of a non-limiting example, the device 102 is optionally operated to insonate the pancreas 106.

In a non-limiting example, the probe is used as part of a method of inducing exfoliation of pancreatic cells or tissue and collecting pancreatic juice that includes such exfoliated cells or tissue for pathologic analysis. In the example, the method includes administering an ultrasound contrast agent that forms microbubbles in a patient's circulatory system prior to insonating the subject. After introducing microbubbles, the organ or tissue, such as the pancreas 106, is subjected to wide area ultrasound energy provided by the probe. The ultrasound application may be described as Low Intensity Non-Focused Ultrasound (LINFU). In the example, the ultrasound energy results in stable cavitation of circulating microbubbles which imparts sufficient energy to the overlying epithelium such that pancreatic cells and, optionally, tissue fragments, disassociate and/or exfoliate. The patient, in the example, is subsequently injected with secretin, a drug that induces pancreatic secretion. Some of the exfoliated and dislodged cells and tissue fragments may be deposited into the pancreatic juice, which is then collected endoscopically.

Example methods of utilizing stable cavitation of circulating microbubbles to induce exfoliation of pancreatic cells and tissue, and optionally collecting such exfoliated cells for pathological evaluation, are described below. Such methods may be used to detect pancreatic cancer, and the method potentially enables detecting the cancer at its earliest stage. The method may be used for periodic screening for pancreatic cancer.

Example methods of inducing sonoporation, by way of a non-limiting example in a target organ such as a pancreas, are described below. Such methods may be used to penetrate desmoplasia to effect drug and/or gene and/or cell extravasation and delivery into tissues (by way of a non-limiting example—tumors).

In some embodiments, sonoporation is effected by ultrasound intensities which cause non-linear oscillations of microbubbles—thus potentially causing microstreaming—which enable transient pores between cells and in cells' membranes—preferable without causing significant and/or noticeable hemorrhage, tissue damage or inflammatory infiltrate.

Non-linear oscillations can be caused by intensities higher than those causing linear, stable oscillations/cavitation, but lower than those causing inertial cavitation which causes implosion and/or shock wave microjets.

An improved transducer device and control system is described herein that is configured to induce stable cavitation of microbubbles potentially in even an entire pancreas simultaneously. The transducer and control system may be calibrated to localize an incidence of inertial cavitation within a target organ and optionally automatically react by lowering the ultrasound energy when such cavitation is detected.

Overview

During insonation, a desired result is producing stable cavitation of microbubbles, in a target organ, and an undesired side effect may potentially be inertial cavitation. Another potential undesired result can be inertial cavitation in tissue which is not the desired target.

In stable cavitation, microbubbles oscillate due to acoustic pressure. In inertial cavitation microbubbles reach resonance size at high power, then collapse, producing microstreams, cavitation nuclei and shockwaves with high temperatures and pressures. When inertial cavitation occurs inside a subject body, the inertial cavitation potentially produces tissue and/or vascular damage.

Inertial cavitation may also potentially cause hemorrhage in small blood vessels such as capillaries and/or small arterioles/venules.

In some embodiments, it is desirable to avoiding causing hemorrhage in the small blood vessels of a target organ such as the pancreas. In some embodiments, insonation is controlled to reduce and/or avoid causing hemorrhage in the small blood vessels.

Controlling Inertial Cavitation

An aspect of some embodiments relates to controlling a LINFU probe, to refrain and/or reduce production of inertial cavitation.

In some embodiments, controlling is limited to refraining and/or reducing production of inertial cavitation within the target organ, for example within the pancreas.

Amplitude or pressure exerted by the LINFU probe becomes lower the further the US signal travels from the LINFU probe.

In some embodiments, the LINFU probe is placed on the skin, and a path from the LINFU probe to the pancreas typically contains fat. In some embodiments, inertial cavitation is allowed in the fat region between the probe and the pancreas, and not allowed deeper than the pancreas, and/or not allowed in the sides of the pancreas.

It is noted that LINFU access to other example organs is contemplated, and the pancreas is described herein as an example organ, which may be insonated from outside a subject's body.

How to Detect Inertial Cavitation

In some embodiments, inertial cavitation is detected by using one or more cavitation detectors, such as passive cavitation detector(s) or other types of hydrophone(s).

When more than one cavitation detectors are used, one or more of a direction and/or a distance of the cavitation from the cavitation detectors are optionally calculated.

In some embodiments, triangulation is used to detect a location of the cavitation, for example by correlation of two or more signals received from two or more cavitation detectors.

Where to Control Inertial Cavitation

In some embodiments, when inertial cavitation is detected within an organ targeted for insonation, a control method is used to reduce and/or eliminate the inertial cavitation within the targeted organ.

In some embodiments, the controlling allows inertial cavitation within a targeted organ.

In some embodiments, the controlling allows inertial cavitation within fatty tissue between a LINFU probe and a targeted organ.

In some embodiments, when inertial cavitation is detected at a depth greater than that of the organ targeted for insonation, a control method is used to reduce and/or eliminate the inertial cavitation.

In some embodiments, when inertial cavitation is detected at an area sideways from the organ targeted for insonation, a control method is used to reduce and/or eliminate the inertial cavitation.

Parameters or Data Input

An aspect of some embodiments relates to which parameters are used to control insonation to refrain and/or reduce production of inertial cavitation in a method or device for exfoliation for cytopathologic cell collection from inside a body.

A non-limiting example list of parameters optionally input to a control unit, the parameters optionally used to determine when and how to control insonation includes:
a transmission center frequency (f0);
acoustic pressure (AP);
a pulse length (PL);
a pulsing interval (PI);
a total insonation time (TIT);
a depth Z beyond which inertial cavitation should be eliminated or reduced, and/or a depth of a top limit of a target organ and a thickness of the organ, and/or a depth of a bottom limit of the organ;
horizontal distance X of a target organ beyond which inertial cavitation should be eliminated or reduced;
vertical distance Y of a target organ beyond which inertial cavitation should be eliminated or reduced;
a target organ for insonation, optionally used to estimate one or more of X, Y and Z;
a qualitative size of a target organ used to estimate one or more of X, Y and Z;
a direction to a center of a target organ used to estimate one or more of X, Y and Z;
an age of a subject, optionally used to estimate one or more of X, Y and Z;
a qualitative description of a subject's physical data, for example “fat”, “obese”, slim” “skinny”, optionally used to estimate depth Z to the target organ; and
a quantitative description of a subject's physical data, for example height, weight, age, Body Mass Index (BMI), optionally used to estimate depth Z to the target organ and/or size of the organ.

In some embodiments, imaging and/or ultrasound imaging is optionally used to determine some parameters for controlling the insonation. A non-limiting list of parameters optionally obtained by imaging includes:
a depth of a target organ;
a thickness of the target organ;
a location with a subject's body of the target organ; and
a location of target organ relative to other subject organs, for example angle and/or direction from tip of sternum to pancreas, distance from tip of sternum, or from skin, to pancreas, and additional geometric measurements.

Control Intensity in Target Organ

An aspect of some embodiments relates to controlling insonation in a target organ.

In some embodiments, the insonation is optionally controlled to provide an approximately uniform intensity in an entire cross-section of a target organ.

In some embodiments, the intensity is described using units of Watts/cm² (Watts per square centimeter).

In some embodiments, obtaining uniform intensity at a cross section of a target organ is optionally achieved by using an array of transducers sized and shaped approximately similarly to the cross section of the target organ, and optionally operating all the transducers with a same insonation signal.

Additional embodiments are described herein, some of which produce an approximately uniform intensity at the cross section by operating at least some of the transducers using insonation signals at different intensities, and/or frequencies, and/or phases.

In some embodiments, the intensity is optionally maintained at as high a value as possible, without inducing inertial cavitation in the target organ.

In some embodiments, the intensity is optionally initially started at some specific high level, and optionally reduced when inertial cavitation is detected, until inertial cavitation is no longer detected.

In some embodiments, the intensity is optionally initially started at some specific high level, directed at a first direction of a first portion of the target organ, and when inertial cavitation is detected in an location where the inertial cavitation is undesired, the energy is optionally steered toward a different portion of the target organ, so that the subject's body is no longer, or much less, insonated.

In some embodiments, controlling the intensity includes controlling and/or adjusting one or more of the above-mentioned center frequency (f0); acoustic pressure (AP); pulse length (PL); pulsing interval (PI); and total insonation time (TIT);

In some embodiments, controlling the intensity includes adjusting an ultrasound pulse duration of the insonation. By way of a non-limiting example, ultrasound pulse duration may start with pulse widths of 20 microseconds, or any other pulse width described herein, and reduce pulse duration when and/or if inertial cavitation is detected in an undesired portion of a subject body.

In some embodiments, controlling the intensity includes adjusting a duty cycle, that is, a ratio between an ultrasound pulse duration and a duration between pulses. By way of a non-limiting example, ultrasound pulse duty cycle may start with a high value, and reduce duty cycle, thereby reducing energy concentration in a subject body, when and/or if inertial cavitation is detected in an undesired portion of the subject body.

In some embodiments, controlling intensity is performed by adjusting all piezo ultrasound transducers' operation in a LINFU probe in a similar fashion—increasing or decreasing intensity to all at once.

In some embodiments, controlling intensity is performed by adjusting two or more groups of piezo ultrasound transducers operations separately. Adjusting the groups separately potentially enables one or more of: steering a direction of insinuation; reducing intensity in one portion of the target organ independently of another portion; and preventing inertial cavitation in a location of the subject body by changing where the insonation intensity is concentrated in the body.

In some embodiments, methods of controlling the ultrasound transducers operation include one or more of:
controlling amplitude of transducers or groups of transducers;
controlling frequency of transducers or groups of transducers;
controlling relative phase between transducers or groups of transducers;
controlling rate of frequency change or frequency sweep of transducers or groups of transducers;
controlling a direction of an ultrasound beam; and
controlling a focus and/or defocus of an ultrasound beam.

Thermal Dissipation from Probe

An aspect of some embodiments relates to thermal dissipation of a LINFU probe used for exfoliation inside a body.

Operating a LINFU probe at intensities which may cause exfoliation may cause a heating of the probe and/or the subject's skin or body.

In some embodiments, the LINFU probe is optionally constructed and/or packaged in a manner, which provides a path for thermal dissipation.

In some embodiments, the LINFU probe optionally includes a heat-dissipating panel.

In some embodiments, the LINFU probe is optionally wrapped with a heat-conducting sheath. In some embodiments, the sheath is filled with heat conducting gel.

In some embodiments, a cooling liquid is optionally circulated within the probe and/or sheath, to provide efficient cooling of the probe.

Structural Features

In some embodiments, the LINFU probe has a shape conforming to a subject's shape, so as to fit over the subject's body and have a defined location relative to a target organ.

By way of a non-limiting example, the LINFU probe may have a shape which fits between left and right ribs, thereby enabling a central placement over the subject's body, at a known height. Such a shape potentially enables placing the LINFU probe at a location suitable for insonating a pancreas, optionally taking advantage of the specific direction from the location to the pancreas typically containing body fat, which may be indifferent to inertial cavitation.

In some embodiments, the LINFU probe is shaped and sized to attach to a subject's body by a strap.

In some embodiments, the strap optionally includes one or more attachment(s) to attach cables for providing power and/or control and/or cooling.

Sonoporation

Solid tumors in some body sites are characterized by a phenomenon known as “desmoplasia”, meaning that a layer of connective tissue develops around the tumor. Desmoplasia inhibits efficacy of therapy by blocking access of chemotherapeutic agent(s) to the tumor. Tumors of the pancreas are particularly desmoplastic and this is one of the reasons that pancreatic cancer is particularly difficult to treat.

Studies have been successfully conducted using ultrasound induced resonance of intravenously administered microbubbles to cause pores to form in the connective tissue surrounding the tumor, allowing the chemotherapy agent to enter. Using ultrasound and microbubbles in this manner is defined as “sonoporation”, using ultrasound to create pores.

In the studies, the ultrasound probe insonator covers a small focused area. The small focused area is thus positioned over a particular tumor. The ultrasound probe is kept fixed on the subject's body, and the subject is kept immobile, for a period of treatment of approximately 30 minutes in order to keep the ultrasound focus area at the tumor. In addition to being inconvenient, use of the small focused probe also limits the effect of sonoporation to small selected areas of tumor. The tumor needs to be detected, its position measured, and the probe kept immobile relative to the tumor for the duration of sonoporation.

An effect of chemotherapy is to treat tumors in the entire organ regardless of their size. Therefore, in some embodiments, use of the LINFU probe in sonoporation potentially enables convenient application of an approximately uniform ultrasonic field to an entire target organ, such as an entire pancreas.

In some embodiments, large area low intensity unfocused ultrasound results in stable cavitation of microbubbles over an entire organ, with the result that the subject does not need to be immobilized. Strapping a LINFU probe to a subject, for example as described herein, allows sonoporation of the subject organ, and in some embodiments, immobilization of the patient is therefore not required. In some embodiments, the patient may potentially be relatively free to move during the insonation period.

In some embodiments, sonoporation may work on vasculature and microvasculature—opening the endothelial layer, either by opening intercellular gaps or cells' membranes, to allow large molecules to pass through.

A potential advantage of wide area insonation as provided by the LINFU probe is an increase in effectiveness of chemotherapy throughout an entire organ simultaneously, promoting destruction of cancerous cells in smaller tumors that may not be visible and/or detectable.

Visualization of Microbubble Resonance

In some embodiments, the LINFU probe as described herein causes resonance of intravenously administered microbubbles in a target organ such as the pancreas. As different patients have different Body Mass Indices (BMIs) and anatomy depending on their age, size and weight, it may be desirable to ensure that the LINFU probe is placed correctly for a specific patient, and that the level of ultrasound insonation is sufficient for that patient.

In some embodiments, visualization is added to a LINFU probe, enabling automated or semi-automated detection that microbubble resonance has been successfully induced in a target organ.

In some embodiments, automated detection of microbubble resonance is optionally performed by image analysis of ultrasound images produced during insonation. In some embodiments, a sonographer optionally detects microbubble resonance in ultrasound images produced during insonation.

In some embodiments, a lower limit on insonation ultrasound energy is determined when microbubble resonance is detected.

In some embodiments, an upper limit on ultrasound energy for a particular patient is determined by detection of inertial cavitation.

In some embodiments, insonation is automatically controlled to be between the lower limit and the upper limit.

In some embodiments, to control that the upper limit is not exceeded, passive cavitation detectors in the LINFU probe monitor for the presence of unwanted inertial cavitation in the target organ, for example in the pancreas.

In some embodiments, to control that the lower limit is met or exceeded, ultrasound visualization elements in the LINFU probe system potentially enable detection of a characteristic pattern produced in ultrasound images by microbubble resonance within the target organ. In some embodiments, presence of the characteristic pattern is optionally confirmed by a sonographer on an ultrasound monitor or can be detected by a trained AI system.

Imaging

In some embodiments, the LINFU probe optionally includes an ultrasound-imaging probe, to image organs and/or tissue, in order to determine location and or depth and/or shape and/or direction to an organ targeted for insonation or of neighboring organs.

In some embodiments, the imaging is optionally performed before insonation for purpose of exfoliation or sonoporation.

In some embodiments, the imaging is performed during insonation for purpose of exfoliation or sonoporation.

In some embodiments, the imaging is performed during insonation for purpose of exfoliation or sonoporation, such that ultrasound imaging is optionally performed during pauses between insonation pulses to visualize the existence of microbubbles in the target organ.

In some embodiments, the imaging is performed during insonation for purpose of exfoliation or sonoporation, such that ultrasound imaging is optionally performed during pauses between insonation pulses.

Example Sonoporation Insonation Parameters

In some embodiments, a transmission center frequency used for in-vivo gene delivery ranges from 0.3 MHz to 14 MHz.

A preferable transmission center frequency may be, for example, 1 MHz, which potentially enables sonoporation using many different types and/or sizes of microbubbles.

In some embodiments, sonoporation insonation uses a pressure of 1 MPa. In some embodiments, the pressure is lowered when the transmission center frequency is decreased.

In some embodiments, a threshold pressure used for extravasation is 0.5 MPa at a frequency of 1 MHz, and MI=0.5.

In some embodiments, a threshold pressure for extravasation is 1.6 MPa at a frequency of 2.25 MHz and MI=1.07.

It is noted that increasing acoustic pressure (AP) potentially enhances a dose delivered during sonoporation, but may potentially also be associated with one or more of hemorrhage, tissue damage, inflammatory infiltrate and a highly heterogeneous distribution within a focal volume.

By way of a non-limiting example, increasing the AP from 1.89 MPa to 4.18 MPa induced a 4-fold increase of human factor IX gene expression.

It is noted that exposure time plays a role in gene delivery when using sonoporation. By way of a non-limiting example, a pressure threshold for brain microvasculature disruption decreased from 0.7 to 0.4 MPa, when PL increased from 0.1 to 10 ms.

In some embodiments, when microbubbles are destroyed within tissue vasculature, pulsing interval is optionally selected to be sufficiently high to allow new microbubbles to replenish.

In some embodiments, a total insonation time increase from 1 minute to 10 minutes induced a 5-fold enhancement of luciferase gene expression—when intensity levels did not cause tissue damage.

In some embodiments, higher and more homogeneous transfection is optionally achieved by using ultrasound probes providing approximately homogenous, large area, ultrasound insonation fields.

In some embodiments, transfection is optionally performed by ultrasound probes with a capability to control direction of the approximately homogenous, large area, ultrasound beam.

Some Example Potential Advantages of Devices and Methods as Described Herein

Regarding Exfoliation

Control of the exfoliation insonation so as to prevent and/or reduce harmful inertial cavitation which may be produced by the intensity of the insonation can potentially enable decreasing time of the exfoliation, potentially improving patient comfort and physician efficiency.

Control of the insonation so as to prevent and/or reduce high temperature at a target organ, or at the subject's skin, or at the probe, potentially improves patient comfort.

Regarding Sonoporation

In some embodiments, sonoporation is optionally used to create pores in overlying desmoplasia allowing for improved exposure of tumor cells to chemotherapeutic agents.

Control of the sonoporation insonation so as to prevent and/or reduce harmful inertial cavitation which may be produced by the intensity of the insonation can potentially enable increasing take-up by increasing intensity, while also preventing damage.

A device as described herein optionally insonates a large area, and does not necessarily require to be fixed in position.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

Before explaining at least one embodiment of the disclosure in detail, it is to be understood that the disclosure is not necessarily limited in its application to the details set forth in the following description or exemplified by the Examples. The disclosure is capable of other embodiments or of being practiced or carried out in various ways.

In some embodiments, an ultrasound transducer is formed of a single crystal that is etched or similarly partitioned to form a series of thin, uniformly arranged transmitting units.

In some embodiments, a probe is configured to deliver sufficient ultrasound radiation to a target organ such as a pancreas in order to impart sufficient energy to the tissue of the target organ, and in some embodiments to a contrast agent flowing within its vasculature, to induce exfoliation of cells and/or tissue.

In some embodiments, the ultrasound transducer is configured to insonate using long pulses, optionally significantly homogenously, an entire organ or a majority of an organ, optionally simultaneously.

In some embodiments, a system includes a probe and connector(s), a belt, an electronic module, a control unit, a processing unit and a Graphics User Interface (GUI).

In some embodiments, the system also includes a disposable sheath that may envelope up to a whole probe and its cable, or part of it, so as to allow the procedure to be sterile, while allowing attachment to the belt that holds the probe in place.

In some embodiments the probe optionally includes a plurality of cavitation detectors and an associated computer system that is configured to i) detect an incidence of cavitation; ii) determine a plane at which the incidence of cavitation is detected; iii) determine if the plane wherein cavitation is detected is located within the target organ; and iv) reduce the intensity of ultrasound energy if the plane where cavitation is detected is on a plane identified as being within the target organ.

Some example technical challenges which are addressed by example embodiments:

Maximizing oscillations of microbubbles, in a form of stable cavitation, without causing inertial cavitation—may be performed by increasing ultrasound pulse duration, and/or duty cycle and/or intensity, and reducing when and/or if inertial cavitation is detected.

Causing the above over a relatively large volume, for example over an entire organ or a sizable portion of an organ—use a relatively large ultrasound probe and/or use the probe to produce a volumetric, unfocused ultrasound beam, optionally an approximately uniform intensity unfocused beam of large aperture.

Causing the above deep within a subject body—enable sufficient ultrasound intensity, to compensate for tissue attenuation and/or enable minimal beam scattering by beam shaping, optionally using a multiple element design of the ultrasound probe.

Adapting the above to different torso sizes—design system to enable increase of intensity while eliminating inertial cavitation at a target organ.

Causing the above without excessive heating of a subject body's skin—providing heat dissipation through a probe backing and/or wrapping the probe with a sheath filled with heat conducting gel and/or reducing insonation intensity.

Maximizing organ coverage—design a large enough 2D probe that produces an approximately homogeneous beam of large enough aperture.

Evading ribs blockage of insonation toward target organ, for example pancreas—place probe below a sternum (and xiphoid process) and enable tilting of beam upwards (Cephalic).

Provide sterility of procedure—use a sterile, optionally disposable, sheath for covering probe and cable. In some embodiments, use a disposable probe. In some embodiments, sterilize probe after each use.

Shorten procedure time—increase intensity and/or duty cycle.

Enable subject comfort—shorten procedure time and/or attach probe to body with a flexible and/or elastic belt and/or reduce probe size.

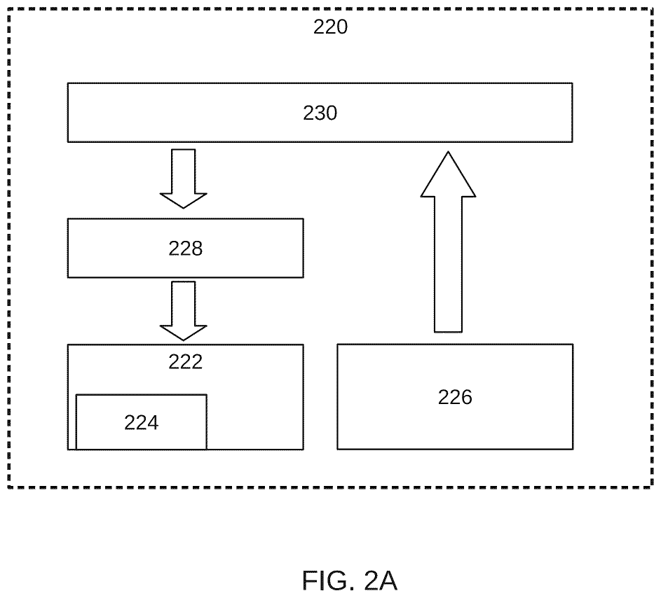

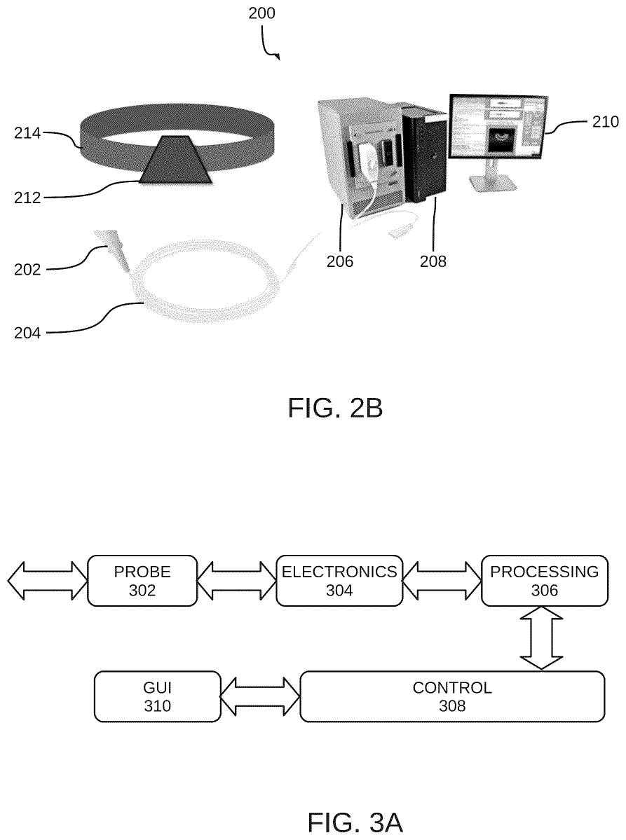

Reference is now made to FIG. 2A, which is a simplified block diagram illustration of a Low Intensity Non-Focused Ultrasound (LINFU) device 220 inducing a wide area of stable microbubble cavitation according to an example embodiment.

The LINFU device 220 of FIG. 2A includes:
an ultrasound probe 222 comprising an ultrasound transducer 224;
a cavitation detector 226;
an electronics unit 228 for adjusting insonation of the ultrasound probe 222; and
a processor 230 for analyzing signals from the cavitation detector 226 and controlling the insonation using the electronics unit 228.

Reference is now made to FIG. 2B, which is a simplified illustration of a system 200 for inducing a wide area of stable microbubble cavitation inside a body according to an example embodiment.

FIG. 2B is intended to show an example system without yet going deeper into details or structure.

FIG. 2B shows an ultrasound probe 202, a cable 204 connecting the probe 202 to an electronic unit 206, which is functionally connected to a computer 208 and a display 210.

FIG. 2B also shows a sheath 212 for attaching or inserting the probe 202 thereto, and a strap 214 to attach the sheath 212 and probe 202 to a subject's body.

The probe 202 is extracorporeal and is used to provide ultrasound insonation within a subject's body at different depths from the skin surface depending on a target organ and anatomy of a subject.

In some embodiments, the system has a capacity to optionally produce a maximal intensity, e.g. of MI≤0.3, approximately uniform throughout a cross-section of a volumetric beam.

The abbreviation MI is used in the present specification and claims for Mechanical Index, defined as peak negative pressure in MPa divided by a square root of frequency of insonation in MHz.

In some embodiments, the system has a capacity to optionally produce an intensity, in a range of 0.01≤MI≤1.9, approximately uniform throughout a cross-section of a volumetric beam.

In some embodiments, when a contrast agent is administered, a lower range of MI may optionally be used, for example 0.01≤MI≤0.3.

In some embodiments, when a contrast agent is not administered, a higher range of MI may optionally be used, for example 0.1≤MI≤1.9.

In some embodiments, by way of a non-limiting example when exfoliation is desired, a lower range of MI may optionally be used, for example 0.03≤MI≤0.3.

In some embodiments, by way of a non-limiting example when sonoporation is desired, a higher range of MI may optionally be used, for example 0.3≤MI≤1.3.

In some embodiments, the contrast agent is administered into the pancreas duct. In some embodiments, such administering may potentially cause unintentional damage to the pancreas, and is used only in cases where a potential danger, pancreas damage, is weighed against potential benefit of ensuring the contrast agent reaches the pancreas duct.

In some embodiments, for example when pancreatic cancer is considered highly likely, the contrast agent is optionally administered into the pancreas duct.

In some embodiments, for example during routine screening, when pancreatic cancer is considered not especially likely, the contrast agent is optionally not administered directly into the pancreas duct.

In some embodiments, for example during routine screening, when pancreatic cancer is considered not especially likely, the contrast agent is optionally not administered at all.

In some embodiments, the system 200 is controlled to refrain from, or reduce, causing inertial cavitation within the target organ.

In some embodiments, the system 200 includes a capacity to detect inertial cavitation and reduce the ultrasound power in a negative feedback manner.

Inertial cavitation that arises from some depths, such as from adipose tissue, is not necessarily harmful and, may optionally not cause change of insonation.

In some embodiments, in order to determine a depth at which inertial cavitation arises, the probe 202 optionally includes a ring of passive cavitation detectors.

In some embodiments, the passive cavitation detectors optionally triangulate a detected inertial cavitation signal to determine depth of the inertial cavitation relative to the probe 202 surface.

In some embodiments, the electronic unit 206 and/or the computer 208 perform the measurements, and/or signal analysis and/or calculations used to determine the depth.

In some embodiments, in the event that cavitation is detected within a plane identified as associated with the target organ, the electronic unit 206 and/or the computer 208 controls the probe 202 producing insonation to prevent unwanted damage to the target organ.

In some embodiments, in the event that inertial cavitation is detected within a plane identified as associated with the target organ, the system 200 optionally automatically reduces the ultrasound power level and/or controls insonation using other methods as described herein, to reduce or eliminate the inertial cavitation.

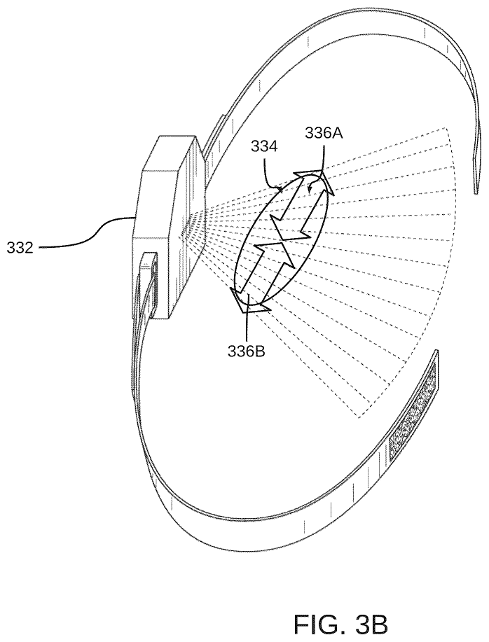

Reference is now made to FIG. 3A, which is a simplified block diagram illustration of a system for Low Intensity Non-Focused Ultrasound (LINFU) for inducing a wide area of stable microbubble cavitation inside a body according to an example embodiment.

FIG. 3A shows a probe 302, in communications with an electronics module 304, in communication with a processing unit 306, in communication with a control module 308, in communication with a GUI module 310.

In some embodiments, the probe 302 optionally includes ultrasound transmitters and/or cavitation detectors.

FIG. 3A is intended to show an optional flow of signals and/or data from the probe 302, optionally to the electronics module 304, optionally to the processing unit 306, optionally to the control module 308, optionally to the GUI module 310, and, optionally, a flow back from an input to the GUI module 310, optionally to the control module 308, optionally to the processing unit 306, optionally to the electronics module 304, optionally to the probe 302.

In some embodiments, the control module 308 is included within the processing unit 306.

In some embodiments, the GUI module 310 is included within the processing unit 306.

In some embodiments, the electronics module 304 is included within the processing unit 306.

In some embodiments, a temperature sensor (not shown in FIG. 3A) is optionally included, to measure temperature of the probe 302.

In some embodiments, the control module 308 may optionally adjusting insonation when and/or if temperature at the probe 302 and or at a subject skin rises above a threshold temperature.

In some embodiments, the processing unit 306 and/or the control module 308 is optionally configured to estimate temperature at a target organ, for example at the pancreas.

Estimating temperature at the target organ may be performed by using a table which correlates ultrasound intensity to temperature, as obtained experimentally in a lab, for subsequent use.

Estimating temperature at the target organ may be performed by using a model of the target organ and subject body and their heat dissipation characteristics.

In some embodiments, the control module 308 may optionally adjusting insonation when and/or if temperature at the target organ rises above a threshold temperature.

Reference is now made to FIG. 3B, which is a simplified illustration of insonating a first portion of a target organ, followed by insonating a second portion of the target organ, according to an example embodiment.

FIG. 3B is intended to demonstrate an optional method of controlling production of inertial cavitation in an undesired portion of tissue, by steering insonation away from a portion where inertial cavitation has been detected.

FIG. 3B shows an example ultrasound probe 332 insonating a first portion 336A of a target organ 334, for example a pancreas 334.

In some embodiments, if and/or when inertial cavitation is detected in the first portion 336A, and/or within a subject body at an undesirable location in the beam used to insonate the first portion 336A, the insonation beam may optionally be steered away from the first portion 336A, for example to insonate a second portion 336B of the target organ 334.

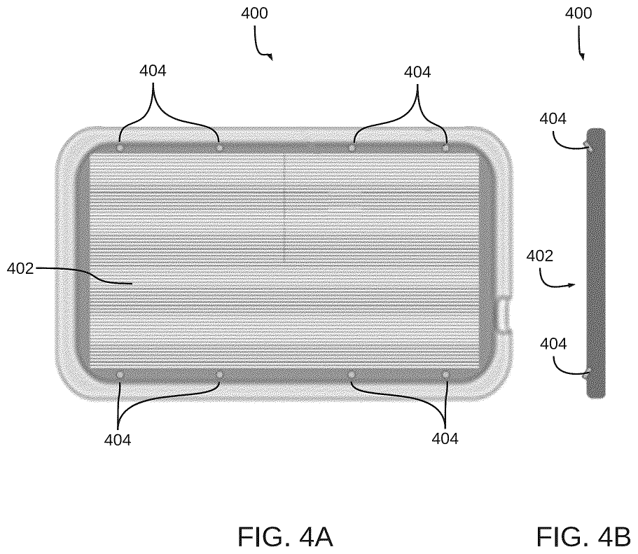

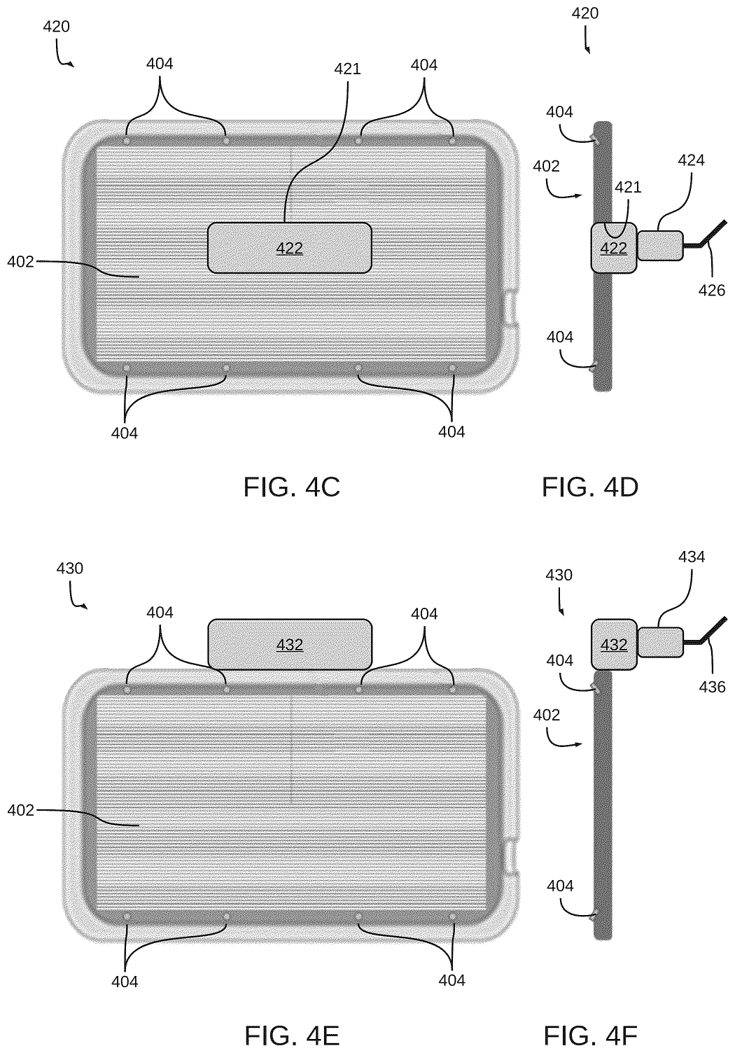

Reference is now made to FIGS. 4A and 4B which are simplified illustrations of an ultrasound probe according to an example embodiment.

FIG. 4A is a front view, and FIG. 4B is a side view, of an ultrasound probe 400.

FIGS. 4A and 4B show the ultrasound probe 400, one or more ultrasound transducers 402, and one or more cavitation sensors 404.

In some embodiments, the probe 400 includes a single crystal that is etched or similarly partitioned to form a series of thin, uniformly arranged ultrasound transmitting units 402.

In embodiments, the probe 400 includes an ultrasound transducer 402 that is configured to provide an approximately uniform field of ultrasonic insonation over an area the size of a human organ, such as, by way of some non-limiting examples, a pancreas, a liver, or a breast.

In embodiments the ultrasound transducer 402 is configured provide pulses of ultrasonic insonation, for example up to 20 microseconds in duration.

In embodiments, the ultrasound transducer 402 is configured to provide pulses of ultrasonic insonation, for example in a range between 20 microseconds up to 400 microseconds in duration.

In some embodiments the ultrasound transducer 402 is constructed of and uses a multiplicity of ultrasounds transducers, such as, for example, long and thin piezoelectric crystals that are arranged, e.g. horizontally or vertically. Such an arrangement potentially enables forming an approximately uniform insonation field, and potentially enables steering away from a line perpendicular to the transducer surface, e.g. cranially or caudally.

In some embodiments, when the ultrasound transducer 402 uses a multiplicity of ultrasound transducers, such as multiple piezoelectric crystals, the ultrasound transducer 402 uses a two-dimensional array of ultrasound transducers.

In some embodiments, the multiplicity of ultrasound transducers are similar or identical transducers.

In some embodiments, the multiplicity of ultrasound transducers is optionally activated at identical frequencies, of, by way of a non-limiting example, in a range of 0.5 MHz to 5 MHz.

In some embodiments, for example to reduce potentially inertial-cavitation-forming relationships among different beams of different crystals within the probe 400, amplitude of the array elements is optionally different between the different crystals.

In some embodiments, for example to reduce potentially inertial-cavitation-forming relationships among different beams of different crystals within the probe 400, amplitude of the array elements is optionally changed over time.

In some embodiments, for example to reduce potentially inertial-cavitation-forming relationships among different beams of different crystals within the probe 400, a rolling frequency is optionally used, where all of the array elements operate at a same frequency at the same time, and the frequency is optionally changed over time.

In some embodiments, for example to reduce potentially inertial-cavitation-forming relationships among different beams of different crystals within the probe 400, a phase difference between different crystals is optionally used. In some embodiments, the phase difference may also be changed over time. In some embodiments, the phase difference may be 5 degrees, 10, 30 up to 90 and even 180 degrees between crystal excitation signals.

In some embodiments, for example to reduce phase relationships among the different beams of different crystals within the probe 400, frequency modulation or phase modulation are optionally employed. In some embodiments, the frequency modulations is optionally performed with a step sizes of e.g. 2 kHz, spanning a range of e.g. +/−200 kHz around a central frequency, varying among neighboring crystals, which together provide an RMS signal that of approximately uniform power.

In some embodiments, a large area of approximately uniform intensity of the ultrasonic beam, optionally similar in size to the organ to be insonated, e.g. the pancreas, is produced by a multiplicity of crystals of various shapes.

In some embodiments, the ultrasonic beam can be optionally steered away from a line perpendicular to the transducer surface, e.g. by +/−150, in order to better penetrate to an organ located under intervening tissue, by way of a non-limiting example under bone.

In some embodiments, the probe potentially enables steering a homogeneous ultrasound beams toward a target organ without readjusting a location of the probe on a subject's body.

In some embodiments, the probe 400 includes one or more temperature sensors (not shown in FIGS. 4A and 4B), which may optionally be used to measure temperature of the probe 400 and/or of a subject's skin (not shown).

In some embodiments, insonation is optionally adjusted when temperature at the probe 400 or at the subject's skin rises above a threshold temperature.

Reference is now made to FIGS. 4C and 4D which are simplified illustrations of an ultrasound probe according to an example embodiment.

FIG. 4B is a front view, and FIG. 4C is a side view, of an ultrasound probe 400.

FIGS. 4C and 4D show the ultrasound probe 420, similar to the ultrasound probe 400 described above with reference to FIGS. 4A and 4B, and further including a center opening 421, for insertion of an imaging probe 422.

Such an embodiment potentially enables guidance of insonation to a target organ, for insonation for causing exfoliation, or for sonoporation, drug delivery etc.

In some embodiments, the imaging probe 422 may optionally be a GE Healthcare E10 probe.

FIG. 4D also shows a handle 424 of the imaging probe 422, and a cable 426 providing power and/or imaging data to and from the imaging probe and one or more units such as the electronics module 304, the processing unit 306, the control module 308, and the GUI module 310 described above with reference to FIG. 3A.

Reference is now made to FIGS. 4E and 4F which are simplified illustrations of an ultrasound probe according to an example embodiment.

FIG. 4E is a front view, and FIG. 4F is a side view, of an ultrasound probe 400.

FIGS. 4F and 4E show the ultrasound probe 430, similar to the ultrasound probe 400 described above with reference to FIGS. 4A and 4B, and further including a side-mounted imaging probe 432.

Such an embodiment potentially enables guidance of insonation to a target organ, for insonation for causing exfoliation, or for sonoporation, drug delivery etc.

In some embodiments, the imaging probe 432 may optionally be a GE Healthcare E10 probe.

FIG. 4F also shows a handle 444 of the imaging probe 432, and a cable 436 providing power and/or imaging data to and from the imaging probe and one or more units such as the electronics module 304, the processing unit 306, the control module 308, and the GUI module 310 described above with reference to FIG. 3A.

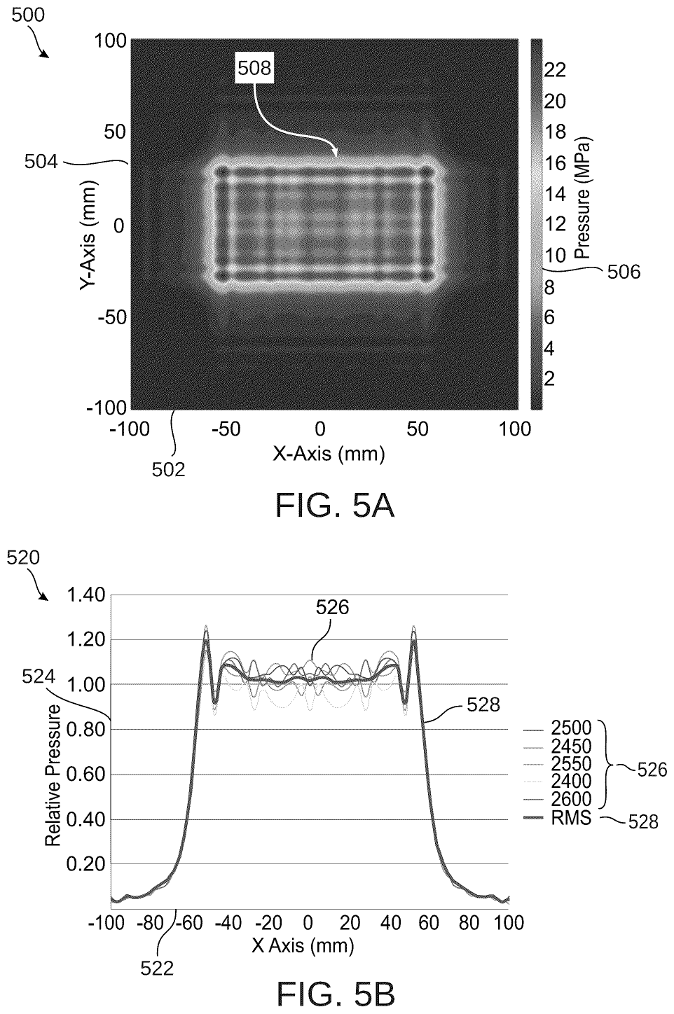

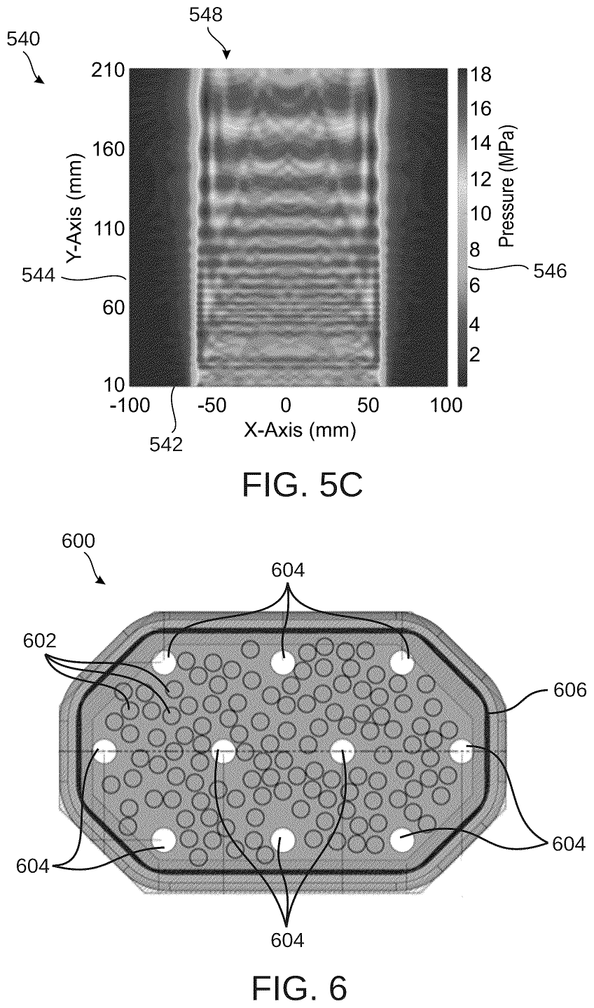

Reference is now made to FIG. 5A, which is a three-dimensional graph of pressure produced by insonation as a function of two-dimensional location at a depth Z in a body, according to an example embodiment.

FIG. 5A shows a graph 500 with an X-axis 502 and a Y-axis 504 in units of millimeters, and a Z-axis of pressure, in units of MPa (Mega Pascal), represented by color, where a color scale 506 is also shown next to the graph 500.

FIG. 5A shows an extent of 120×70 millimeters with an approximately uniform insonation pressure of 14-24 MPa, surrounded by an area which is much less insonated, if at all.

Reference is now made to FIG. 5B, which is a graph of pressure produced by insonation as a function of one-dimensional location, according to an example embodiment.

FIG. 5B shows a graph 520 with an X-axis 522 in units of millimeters, and a Y-axis 524 of relative pressure, in units normalized to 1.0.

FIG. 5B shows five normalized insonation pressure lines 526 at frequencies of 2,400 2,450 2,500 2,550 and 2,600 kHz, produced as a sequence of a rolling frequency sweep from 2.4 MHz to 2.6 MHz in steps of 50 kHz, and one line 528 of normalized Root Mean Square (RMS) pressure.

FIG. 5B shows an extent of 120 millimeters with an approximately uniform relative insonation pressure of 1, surrounded by an area which is much less insonated, if at all.

Reference is now made to FIG. 5C, which is a three-dimensional graph of pressure produced by insonation as a function of depth Z in a body, according to an example embodiment.

FIG. 5C shows a graph 540 with an X-axis 542 and a Z-axis 544 in units of millimeters, and pressure, in units of MPa, represented by color, where a color scale 546 is also shown next to the graph 540.

FIG. 5C shows an extent of 120 millimeters breadth (X-axis 542) by 200 millimeters depth (Z-axis 544) with an approximately uniform insonation pressure of 14-24 MPa, with an area on each side which is much less insonated, if at all.

In some embodiments, the probe enables achieving a large field of simultaneous and approximately homogenous or approximately uniform insonation by using a multiplicity of identical, or similar, piezoelectric crystals, scattered randomly over the surface of the probe.

Reference is now made to FIG. 6, which is a simplified illustration of an ultrasound probe with a random scattering of ultrasound transducers according to an example embodiment.

FIG. 6 shows an ultrasound probe 600 with a random scattering of ultrasound transducers 620, for example piezoelectric crystals, on a face of the probe 600.

In some embodiments, the ultrasound probe 600 includes cavitation sensors 604 placed on the face of the probe 600.

In some embodiments, the ultrasound probe 600 includes a seal 606 surrounding the face of the probe 600.

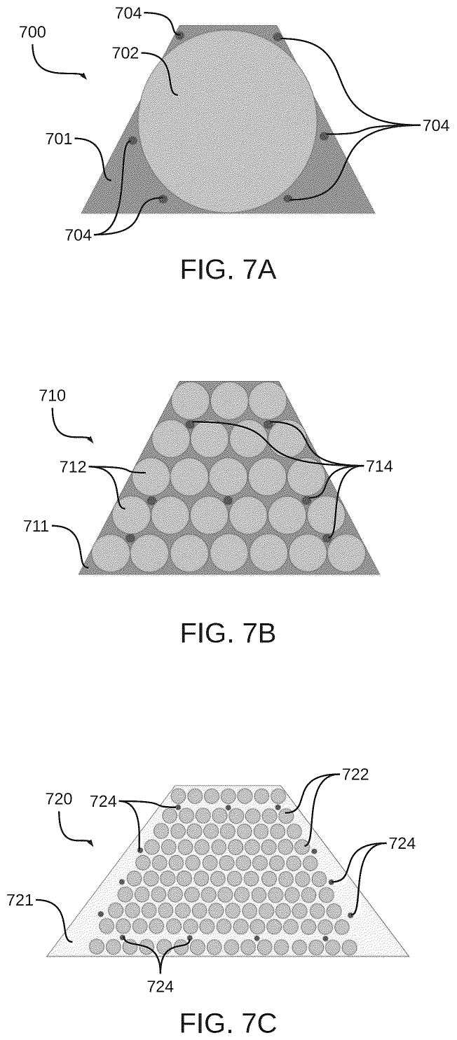

Reference is now made to FIGS. 7A, 7B and 7C, which are simplified illustrations of ultrasound probes with an arrangement of ultrasound transducers and cavitation detectors according to example embodiments.

FIG. 7A shows an ultrasound probe 700 with one ultrasound transducer 702, and several cavitation detectors 704 placed on the face of the probe 700.

FIG. 7A shows 1 ultrasound transducer, a piezo disc element, capable of insonation at a range of 2-3 MHz, and 6 passive cavitation detectors 704.

FIG. 7B shows an ultrasound probe 710 with several ultrasound transducers 712 arranged on a face of the probe 710, and also several cavitation detectors 714 placed on the face of the probe 710.

FIG. 7B shows 25 ultrasound transducers, piezo disc elements, capable of insonation at a range of 2-3 MHz, and 7 passive cavitation detectors 704.

FIG. 7C shows an ultrasound probe 720 with several ultrasound transducers 722 arranged on a face of the probe 720, and also several cavitation detectors 724 placed on the face of the probe 720.

FIG. 7C shows 115 ultrasound transducers, piezo disc elements, and 13 cavitation detectors 704.

In some embodiments, the probe 700 is shaped as a trapeze, with a long base width of, by way of a non-limiting example, 13.5 centimeters, a short base width of 5 centimeters, and a height of 10 centimeters.

FIGS. 6 and 7A-7C show various arrangements of ultrasound transducers 722. The various arrangements may be used to achieve insonation in a target organ, to maintain an insonation pressure sufficient to produce exfoliation in the target organ, while eliminating or at least reducing occurrence of pressure peaks that may produce inertial cavitation.

FIGS. 6 and 7A-7C show various arrangements of cavitation sensors. The various arrangements may be used to detect inertial cavitation, in order to provide input to controlling ultrasound insonation, using one or more of the methods of controlling ultrasound insonation as listed herein, to reduce or eliminate inertial cavitation.

FIGS. 6 and 7A-7C show various shapes of the example embodiment probes. It is noted that in some embodiments, the probes may be shaped to fit on a subject's body, for example between left and right ribs, around an arm, around a leg. Such shapes can potentially provide a known location of the probe relative to a target organ.

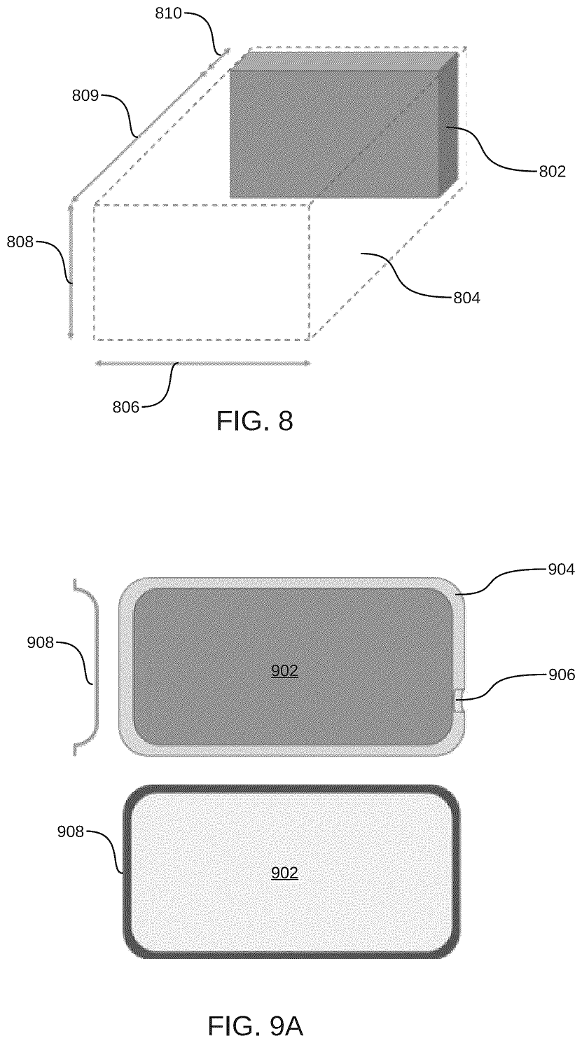

Reference is now made to FIG. 8, which is a simplified illustration of a volume of insonation according to an example embodiment.

FIG. 8 shows a volume of target organ 802, marked by a solid line, a volume of intervening tissue 804, marked by a dashed line.

By way of a non-limiting example, the target organ 802 is a pancreas, with a width 806 of 10-12 centimeters, a height 808 of 8 centimeters, and a depth 810 of 2-4 centimeters.

By way of a non-limiting example, when the target organ 802 is a pancreas, the intervening tissue may have a width 806 of 10-12 centimeters, a height 808 of 8 centimeters, and a depth 809 of 12-14 centimeters.

It is noted that a depth of intervening tissue may be different for different cases: smaller for thin subjects, or young subjects, or small subjects, larger for fat subjects, or older subjects, or larger subjects.

It is noted that dimensions of the target organ may vary, depending on which target organ and/or on the subject.

It is noted that dimensions of the intervening tissue may vary, depending on which target organ and/or on physical dimensions of the subject.

Additional Features

In some embodiments, a large area, when compared to focused ultrasound, and/or a large volume, when compared to focused ultrasound, is insonated. For example, in case of insonating a pancreas, an area of 60-160 cm 2, or a volume as shown in FIG. 8, is optionally insonated by relatively long pulses, for example pulses in a range of 20 microseconds to 400 microseconds.

In some embodiments, dissipation of heat produced by the probe is optionally enabled by attaching or constructing a heat-dissipating back panel for the probe.

In some embodiments, dissipation of heat produced by the probe is optionally enabled by wrapping a back and/or sides of the probe with a heat-conducting sheath, containing heat-conducting gel.

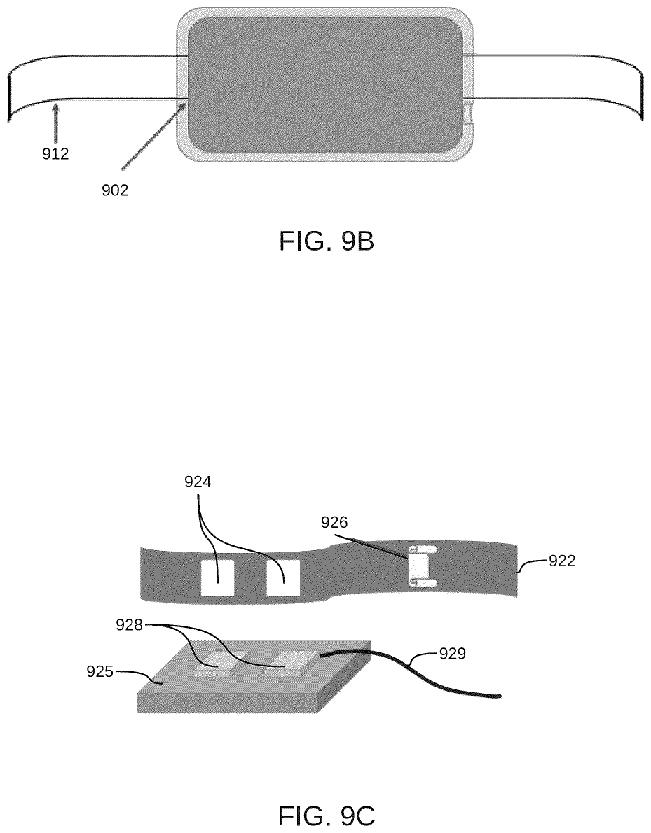

Reference is now made to FIG. 9A, which is a simplified illustration of a heat conducting cover for an ultrasound probe according to an example embodiment.

The top-right drawing in FIG. 9A shows a view from a bottom of a heat conducting cover 902, the side that the probe will optionally touch. Also shown is an optional recess 906 in the cover, to enable cables to pass through to the probe.

In some embodiments, the side the probe will optionally touch may include heat-conducting gel. In some embodiments, the side the probe will optionally touch may include a metal heat conductor.

The top-left drawing in FIG. 9A shows a side, cross-sectional view of an optional magnetic strip 908 or panel 908 which may be attached to the heat conducting cover, potentially enabling magnetic attachment of the cover to the probe.

The bottom drawing in FIG. 9A shows a view from a bottom of the heat conducting cover 902, with the magnetic strip 908 or panel 908 appearing around a circumference of the cover 902.

Reference is now made to FIG. 9B, which is a simplified illustration of a heat conducting cover and an attachment belt for an ultrasound probe according to an example embodiment.

FIG. 9B shows an optional belt 912 attached to a heat conducting cover 902.

The belt may be sized for attachment to a torso, or to other body parts, depending on a location of the target organ.

Reference is now made to FIG. 9C, which is a simplified illustration of an attachment belt and an ultrasound probe or probe cover according to an example embodiment.

FIG. 9C shows a belt 922 with one or more holes 924, located to fit one or more corresponding protuberances 928 on a probe 925 or a probe cover 925.

FIG. 9C also shows a power and/or data cable 929 leading to and from the probe 925 or a probe cover 925.

FIG. 9C also shows an optional attachment point 926 such as a small belt 926, optionally a Velcro attachment strip, which may optionally be used to attach the cable 929 to the belt 922, or a sheath with ultrasound gel.

FIG. 9C also shows an optional attachment point 926 such as a small belt 926, optionally a Velcro attachment strip, which may optionally be used to attach the cable 929 to the belt 922.

In some embodiments, a cooling liquid is optionally circulated within the probe and/or cover and/or sheath, to provide efficient cooling of the probe.

In some embodiments, a sheath assembly is designed to provide a single use, ultrasound gel filled, sterile barrier between the patient and the transducer surface, and/or to keep the transducer in place during duration of insonation, and/or to deflect heat away from the patient's skin.

In some embodiments, the sheath assembly holds the transducer at a prescribed location by being connected to a single use disposable belt that is placed around the abdomen and contains a port to allow connection of a non-sterile cable from the transducer to its electronic controller without compromising sterility.

A Non-Limiting Example Embodiment of a Method for Safely Generating a Wide Area of Stable Microbubble Cavitation within the Body

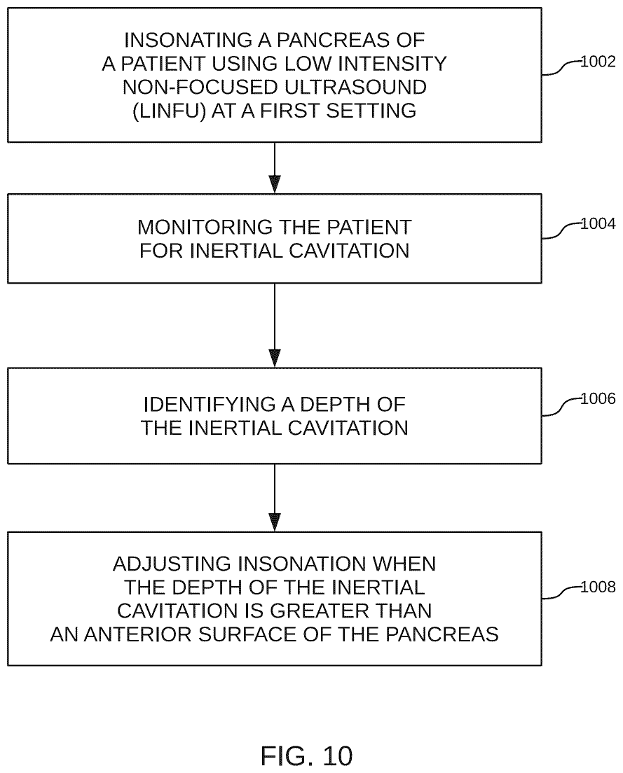

Reference is now made to FIG. 10, which is a simplified flow chart illustration of a method for exfoliating a pancreas according to an example embodiment.

The method of FIG. 10 includes:
insonating a pancreas of a patient using Low Intensity Non-Focused Ultrasound (LINFU) at a first setting (1002);
monitoring the patient for inertial cavitation (1004);
identifying a depth of the inertial cavitation (1006);
adjusting insonation when the depth of the inertial cavitation is greater than an anterior surface of the pancreas (1008).

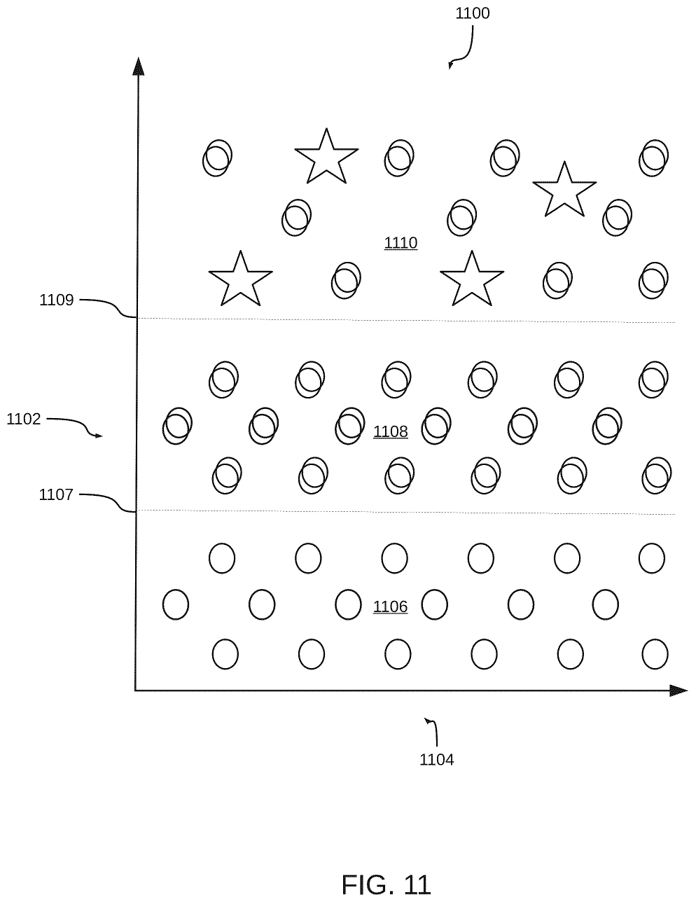

It is noted that other internal organs may be exfoliated similarly Reference is now made to FIG. 11, which is a simplified illustration of controlling a level of insonation according to an example embodiment.

FIG. 11 shows a graph 1100 with a Y-axis 1102 showing a qualitative level of insonation.

FIG. 11 shows a lower limit 1107, below which 1106 microbubble resonance is not detected, and above which 1108 microbubble resonance is detected, and an upper limit above which 1110 inertial cavitation is detected.

In some embodiments, a level of insonation is optionally controlled to be between the lower limit 1107 and the upper limit 1109, using at least one of several methods described herein.

It is expected that during the life of a patent maturing from this application many relevant ultrasound transducers will be developed and the scope of the term ultrasound transducer is intended to include all such new technologies a priori.

It is expected that during the life of a patent maturing from this application many relevant cavitation detectors will be developed and the scope of the term cavitation detector is intended to include all such new technologies a priori.

As used herein with reference to quantity or value, the term “approximately” means “within ±25% of”.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for producing an organ-sized area of stable microbubble cavitation comprising:
insonating an organ of a patient using Low Intensity Non-Focused Ultrasound (LINFU) at a first setting;
monitoring the organ to detect presence of desired stable cavitation microbubble resonance and presence of unwanted inertial cavitation;
when the presence of stable cavitation microbubble resonance is not detected then adjusting insonation parameters so as to increase the level of insonation; and
when inertial cavitation is detected then adjusting the insonation parameters so as to decrease the level of insonation.

2. The method according to claim 1, wherein monitoring the pancreas comprises:
producing ultrasound images of the pancreas; and
monitoring for microbubble resonance by detecting the microbubble resonance in the ultrasound images.

3. The method according to claim 2, wherein detecting the microbubble resonance in the ultrasound images comprises performing image analysis of the ultrasound images.

4. The method according to claim 2, wherein monitoring the pancreas comprises monitoring for inertial cavitation by cavitation detectors.

5. The method according to claim 1, wherein: the insonating comprises insonating a pancreas of a patient using Low-Intensity Non-Focused Ultrasound (LINFU) at a first setting, and; the monitoring comprises monitoring the patient for inertial cavitation; and the method further comprises:
identifying a depth of the inertial cavitation; and
automatically adjusting insonation when the depth of the inertial cavitation is greater than an anterior surface of the pancreas.

6. The method according claim 5, wherein the adjusting insonation comprises controlling insonation to avoid causing tissue damage.

7. The method according to claim 5, wherein:
the insonating the pancreas of a patient comprises insonating a first portion of the pancreas; and
the automatically adjusting insonation comprises steering the insonation to a second, different, portion of the pancreas.

8. The method according to claim 1, comprising:
insonating a pancreas of a patient using Low Intensity Non-Focused Ultrasound (LINFU) at a first setting;
determining temperature produced by the insonating;
automatically adjusting insonation when the temperature exceeds a threshold temperature.

9. A Low Intensity Non-Focused Ultrasound (LINFU) device comprising:
an ultrasound probe comprising an ultrasound transducer;
a cavitation detector;
an electronics unit for adjusting insonation of the ultrasound probe; and
a processor for analyzing signals from the cavitation detector and controlling the insonation using the electronics unit, wherein the processor is configured to adjust insonation parameters so as to increase the level of insonation when the presence of stable cavitation microbubble resonance is not detected, and to adjust the insonation parameters so as to decrease the level of insonation when inertial cavitation is detected.

10. The device according to claim 9, wherein the transducer is configured to provide an approximately uniform field of ultrasonic insonation over an area the size of a human organ, wherein the human organ is a pancreas.

11. The device according to claim 9, wherein the probe is shaped to fit between a patient's ribs, below the patient's sternum.

12. The device according to claim 9, wherein the processor is configured to determine a depth of cavitation detected by the cavitation detector.

13. The device according to claim 12, wherein the processor is configured to determine a three dimensional location of cavitation detected by the cavitation detector.

14. The device according to claim 13, and further comprising a component for removing heat from the ultrasound probe.

15. The device according to claim 9, and further comprising a belt for attaching to a subject's body.

16. The device according to claim 15, and further comprising a temperature sensor for measuring temperature at a subject's body.

17. The device according to claim 16, and further comprising a temperature sensor for measuring temperature at the ultrasound probe.

18. A system for producing an organ-sized area of stable microbubble cavitation comprising:
a device according to claim 9; and
a user interface configured for entering parameters related to producing stable microbubble resonance while avoiding inertial cavitation.

19. The system according to claim 18, wherein the user interface is configured for entering physical parameters related to a subject planned for exfoliation.

20. The system according to claim 18, wherein the user interface is configured for entering physical parameters related to a subject planned for sonoporation.

21. The system according to claim 18, wherein the system includes communication with medical database for obtaining subject data.
